(19) United States
(12) Patent Application Publication
(10) Pub. No.: US 2026/0199619 A1
(43) Pub. Date: Jul. 16, 2026

(54) OXYGENATING OROPHARYNGEAL AIRWAY DEVICES, KITS RELATING TO SAME, AND METHODS OF USING SAME

(71) Applicant: BAYLOR RESEARCH INSTITUTE D/B/A BAYLOR SCOTT & WHITE RESEARCH INSTITUTE, Dallas, TX (US)

(72) Inventor: Jeramie B. HANSON, Belton, TX (US)

(21) Appl. No.: 19/136,474

(22) PCT Filed: Dec. 6, 2023
(86) PCT No.: PCT/US2023/082631

§ 371 Date: Jun. 6, 2025

Related U.S. Application Data
(60) Provisional application No. 63/386,396, filed on Dec. 7, 2022.

(51) Int. Cl. A61M 16/04; A61M 16/08

(52) U.S. Cl. CPC A61M 16/0493; A61M 16/0816; A61M 2210/0625

(57) ABSTRACT

Oxygenating oropharyngeal airway devices, kits including at least such devices, and methods of using such devices. Such a device may be inserted into a patient in need of airway management and used to oxygenate the patient through a lumen of the device both before and during any needed intubation, including by direct laryngoscopy. Another lumen of the device may be used to monitor end tidal carbon dioxide. Another lumen of the device may be used to insert a scope into a patient to visualize the airway. Ventilation, achieved by manipulating a bag coupled to a mask adequately sealed over the patient's nose and mouth, may be performed while one of the devices is positioned in the patient's mouth and while the patient is receiving oxygen from tubing running under the mask and attached to the device and an oxygen source.

This application claims the benefit of priority of U.S. Provisional Patent Application No. 63/386,396, filed Dec. 7, 2022, which is hereby incorporated by reference in its entirety.

### FIELD OF INVENTION

The invention relates generally to oral airway devices that can be inserted into the airway of a living being for the purpose of facilitating oxygenation. The invention also relates to kits that include one or more such devices, and to methods of using such devices.

### BACKGROUND

High-flow nasal cannula (HFNC) oxygen therapy was initially introduced in the early 2000s for management of apnea in neonates and has recently gained favor in the adult operating room and critical care settings. Through anatomic dead space washout and improved positive end expiratory pressure, HFNC oxygen therapy can effectively allow maintenance of normal oxygen saturations in apneic patients for prolonged periods of time. HFNC oxygen therapy does, however, have several limiting factors. First, it requires additional equipment, including an oxygen heater, a humidifier, a high-flow oxygen regulator, and a nasal cannula designed for this purpose. Additionally, HFNC oxygen therapy is relatively inefficient, requiring oxygen flow rates up to sixty (60) liters per minute. Finally, because of the additional equipment required, HFNC oxygen therapy is not ideal in emergent situations.

### SUMMARY OF THE INVENTION

Embodiments of the present oxygenating oropharyngeal airway devices, which may be referred to as oral airway devices, can be used to provide relatively high-flow oxygen orally, directly above the glottis. Supraglottic oxygenation with embodiments of the present oral airway devices may be achieved during ventilation and during intubation. It is believed that, by bypassing the dead space and soft tissue obstruction of the nasopharynx using one of these embodiments, maintenance of normal oxygen saturation can be achieved at oxygen flow rates lower than sixty liters per minute (in at least some circumstances) and without the need for an oxygen heater, a humidifier, or a high-flow oxygen regulator. Embodiments of the present oral airway devices include a connector configured to be coupled to an oxygen source (such as a wall oxygen source or an e-cylinder), making them suitable for the operating room, emergency department, and critical-care settings. That connector is in fluid communication with a lumen within the oral airway device through which oxygen from an oxygen source may be directed to provide apneic oxygenation to the patient.

The present oral airway devices may be used in place of a conventional “Guedel” oropharyngeal airway. Embodiments of the present oral airway devices include two body components, which are configured to be coupled together before insertion into a patient's mouth (such as after manufacturing and prior to packaging for shipment, but at a minimum prior to insertion into a patient's mouth) and uncoupled from each other, including while at least a portion of each body component is (still) positioned in the patient's mouth. One of the two body components is configured for oxygenation and includes at least one lumen for delivering oxygen (therethrough) to a patient from an oxygen source coupled thereto, and may also include a second lumen (distinct from, and not in fluid communication with, the oxygen lumen) for monitoring end tidal carbon dioxide (sometimes referred to as ETCO2 for ease of reference) by coupling the second lumen to an ETCO2 monitor. The other of the two body components is shaped to have a longitudinal open channel that, when the two body components are coupled together, forms a third lumen large enough to accommodate a fiber optic scope that can be inserted into the third lumen and directed to a target location within the patient's airway; an endotracheal tube can ultimately be placed over such a scope during intubation and after the non-oxygenating body component has been removed from the patient's mouth. Thus, the oxygenating body component of the present oral airway devices may be used to provide supraglottic oxygenation to a patient while the patient is being intubated, either via direct laryngoscopy or with a fiber optic scope; such procedures comprise embodiments of the present methods. In such method embodiments involving direct laryngoscopy, a practitioner may position an embodiment of the present oral airway devices that has been coupled to an oxygen source in a patient's mouth, and, as oxygen is delivered through the first (oxygenating) lumen of the device, intubate the patient via direct laryngoscopy. For example, in such method embodiments, when the practitioner is ready to intubate the patient after establishing an airway using one of the present oral airway devices, and while oxygenating the patient through the first lumen of the device and/or monitoring ETCO2 via the second lumen of the device, the practitioner may decouple the body components of the device from each other (while a portion of each remains in the patient's mouth), remove the non-oxygenating body component from the patient's mouth, and then perform direct laryngoscopy on the patient while a portion of the oxygenating body component remains within the patient; more specifically, while a portion of the oxygenating body component remains within the patient (including, e.g., after the practitioner has shifted it to the left side of the patient's mouth to increase room for a laryngoscope and an endotracheal tube) and the patient is receiving supraglottic oxygenation through the first lumen of the oxygenating body component, the practitioner may insert the blade of a laryngoscope into the patient's mouth and, once the blade is positioned as desired, insert an endotracheal tube into the patient such that the distal end thereof is in a desired position in the patient's trachea. In other method embodiments involving fiber optic scope-supported intubation, a practitioner may position an embodiment of the present oral airway devices that has been coupled to an oxygen source in a patient's mouth, and, as oxygen is delivered through the first lumen of the device, intubate the patient using a fiber optic scope, which a practitioner can use to visualize a portion of the patient's anatomy (e.g., the patient's airway and vocal cords). For example, in such method embodiments, when the practitioner is ready to intubate the patient after establishing an airway using one of the present oral airway devices, and while oxygenating the patient through the first lumen of the device and/or monitoring ETCO2 via the second lumen of the device, the practitioner may insert a scope (e.g., a fiber optic scope, which can be used to visualize the patient's airway) into the third lumen of the device and navigate its distal portion to an appropriate location in the patient; the practitioner may then decouple the body components of the device from each other (while a portion of each remains in the patient's mouth), remove the non-oxygenating body component from the patient's mouth while portions of the oxygenating body component and the scope remain within the patient (including, e.g., after the practitioner has shifted the oxygenating body component to the left side of the patient's mouth), and then advance an endotracheal tube over the scope to a desired location (while still oxygenating the patient through the first lumen and, optionally, monitoring ETCO2 via the second lumen) to intubate the patient. In some embodiments of the present oral airway devices, the oxygenating body component includes grooves and the non-oxygenating body component includes connector tabs, each with a portion shaped to complement the shape of one of the grooves, such that a portion of a given tab fits within a given groove when the body components are coupled together; in other embodiments, the grooves and tabs are on the opposite body components.

Embodiments of the present oral airway devices include a connector, in fluid communication with the second lumen, that can be configured as a port connectable to a tube that can be used as a carbon dioxide (CO₂) sample line to monitor ETCO2.

Embodiments of the present oral airway devices can be utilized in a variety of different situations and deployed quickly in urgent and emergency situations. The present oral airway devices can serve to oxygenate patients requiring cardio pulmonary resuscitation (CPR), thereby limiting interruptions in chest compressions in the non-intubated patient and enabling the monitoring of ETCO2, which will reflect whether quality chest compressions are being performed. The present oral airway devices may also serve as an effective emergency airway adjunct in situations where a patient cannot be ventilated.

Embodiments of the present methods include the method embodiments described above and, more broadly, any one or more of the uses of an embodiment of the present oral airway devices described above. Accordingly, in some embodiments of the present methods, one of the present oral airway devices may be inserted into a patient's mouth, including a patient undergoing CPR or who undergoes CPR after such insertion. In some embodiments of the present methods, oxygen may be delivered to a patient at a rate of five (5) to twenty-five (25) liters per minute (including every measurable increment of liters per minute between five and twenty-five, including increments of one) through one of the present oral airway devices that has been inserted into the patient's mouth. In some embodiments of the present methods, one of the present oral airway devices may be used to apply positive end-expiratory pressure (PEEP) to a patient to prevent, reverse, or improve atelectasis. The present oral airway devices are configured such that, when the device is connected to a tube (that is connected to an oxygen source) and/or to a tube that is connected to equipment for monitoring end tidal carbon dioxide, a conventional mask that can be used for bag-valve-mask ventilation may be placed over the patient's nose and mouth and adequately sealed; as a result, some embodiments of the present methods include placing a mask over the patient's nose and mouth while one of the present devices (connected, or not, to one or more tubes) is inserted in the patient's mouth, and ventilating the patient using a bag coupled to the mask.

The present kits may include any one or more of: one of the present oral airway devices; and a length of oxygen tubing with connectors, one of which is configured for connection to an oxygen source and the other of which is configured for connection to the oxygen connector of the included one of the present oral airway devices (such as to the female oxygen tubing connector on a shorter length of oxygen tubing attached to the oxygen connector of the included one of the present oral airway devices). At least some embodiments of the present kits includes a container, such as a box or a pouch, that is sealed and includes any one or more of the foregoing components. In at least some embodiments of the present kits that include one of the present oral airway devices, the airway device may have a color that reflect its size (e.g., pink for an infant). The items in such a kit may be made from materials that render them suited for a single-use application.

The term “coupled” is defined as connected, although not necessarily directly, and not necessarily mechanically. The terms “a” and “an” are defined as one or more unless this disclosure explicitly requires otherwise. The term “substantially” is defined as largely but not necessarily wholly what is specified (and includes what is specified; e.g., substantially perpendicular includes perpendicular), as understood by a person of ordinary skill in the art. In any disclosed embodiment, the term “substantially” or “approximately” can be substituted with “within [a percentage] of” what is specified, where the percentage includes 0.1, 1, 5, and up to 10 percent.

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, something that “comprises,” “has,” “includes,” or “contains” one or more elements possesses those one or more elements, but is not limited to possessing only those one or more elements. Likewise, a method that “comprises,” “has,” “includes,” or “contains” one or more steps possesses those one or more steps, but is not limited to possessing only those one or more steps.

Any embodiment of any of the disclosed oral airway devices and kits and methods can consist of or consist essentially of—rather than comprise/have/include/contain—any of the described elements, features, and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

Further, an oral airway device or portion thereof that is configured in a certain way is configured in at least that way, but it can also be configured in a way or ways other than those specifically described.

The feature or features of one embodiment may be applied to other embodiments, even though not described or illustrated, unless expressly prohibited by this disclosure or the nature of the embodiments.

Some details associated with some of the embodiments of the present oral airway devices, the present kits, and the present methods are described above, and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears, and every reference number in a given figure is not always described. Identical reference numbers do not necessarily indicate an identical structure. Rather, the same reference number may be used to indicate a similar feature or a feature with similar functionality, as may non-identical reference numbers. The devices in FIGS. 1-12 are drawn to scale, meaning the sizes of the depicted elements are accurate relative to each other for at least the depicted embodiments of the present oral airway devices; but as a person of ordinary skill in the art will understand, this invention is not limited to the depicted embodiments or usage of any of them, and other embodiments that do not possess the depicted sizes also fall within the scope of the claims. The devices shown in the figures were drawn (or prototyped, for those shown in FIGS. 13-15) using Shapr3D software and, as a result, include many lines that, as those of ordinary skill in the art will understand, do not represent “hard” changes of angle. Instead, such lines may respectively show where a rounded section meets a flat, or straight, section. Additionally, some lines are extraneous (or show relatively small features or shapes that are extraneous) to the depicted device and are generally the result of expediently generating a three-dimensional printed prototype. The relevant figures were created using the “orthographic” (rather than the “perspective”) “appearance” setting of the software.

FIG. 1 is a perspective view of one embodiment of the present oral airway devices.

FIG. 2 is a perspective view of the device shown in FIG. 1.

FIG. 3 is a front view of the device shown in FIGS. 1 and 2.

FIG. 4 is a back view of the device shown in FIGS. 1 and 2.

FIG. 5A and FIG. 5B are right and left side views, respectively, of the device shown in FIGS. 1 and 2.

FIG. 6 shows an enlarged detail perspective view of the distal end of the body of the device shown in FIGS. 1 and 2.

FIG. 7 shows an enlarged detail perspective view of a proximal portion of the device shown in FIGS. 1 and 2.

FIG. 8 shows one embodiment of the present oral airway devices with a first length of oxygen tubing connected to the device's oxygen connector; a female oxygen tubing connector—to which an oxygen source (not shown) may be connected via a second length of oxygen tubing (not shown)—is shown attached to the free end of the first length of oxygen tubing.

FIG. 9 is a perspective view of the device shown in FIGS. 1 and 2 in which the first body component and second body component thereof are not coupled to each other.

FIG. 10 is a perspective view showing the second body component of the device shown in FIGS. 1 and 2.

FIG. 11 is a perspective view showing the first body component of the device shown in FIGS. 1 and 2.

FIG. 12 is a cross-sectional view of the first body component of the device shown in FIGS. 1 and 2 taken across a plane that is parallel to the straight section of the body thereof, showing an interior of the connector and a portion of the related lumen through which ETCO2 may be monitored and further showing an interior of the connector and a portion of the related lumen through which oxygen may be delivered.

FIG. 13 shows a mask usable with a bag-valve-mask ventilation system being placed over a mannequin that has one of the present oral airway devices inserted in the mannequin's mouth.

FIGS. 14 and 15 show the mask from FIG. 13 in a sealed positioned over the depicted oral airway device and the length of tubing attached to a connector thereof, with the length of tubing positioned so that one portion is under the mask and another portion is outside the mask, with the mask adequately sealing for ventilation thereover.

FIG. 16 is a diagram showing the components of one embodiment of the present kits.

### DETAILED DESCRIPTION

The present oral airway devices are designed for use in a variety of airway-management circumstances, including those involving CPR. The present oral airway devices are also configured so that, while a given one of the devices is still in the patient's mouth, the device can be separated into two body components-the non-oxygenating one of which may be removed from the patient's mouth, and the oxygenating one of which may remain in the patient's mouth, facilitating oxygenation of the patient, including during any subsequent intubation. During use of at least some embodiments of the present oral airway devices, oxygen may be delivered to the patient without the need for the type of equipment typically used in HFNC oxygen therapy and (it is believed) at lower flow rates than are typically associated with such therapy. Furthermore, using one of the present oral airway devices during CPR can allow a non-intubated patient to by oxygenated as chest compressions are performed and, when the device is connected to an ETCO2 monitor, provide evidence of the quality of those compressions.

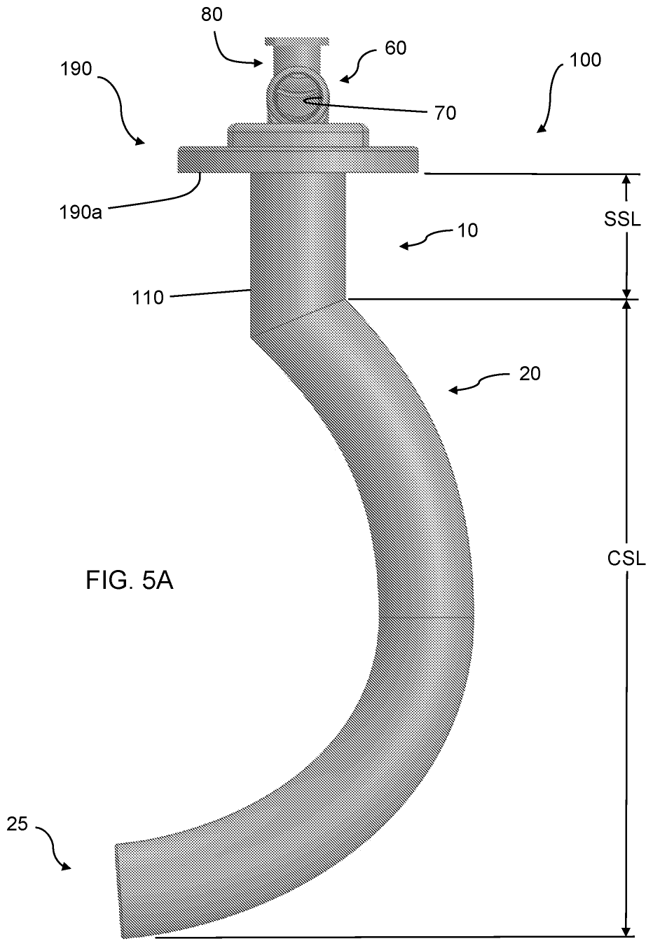

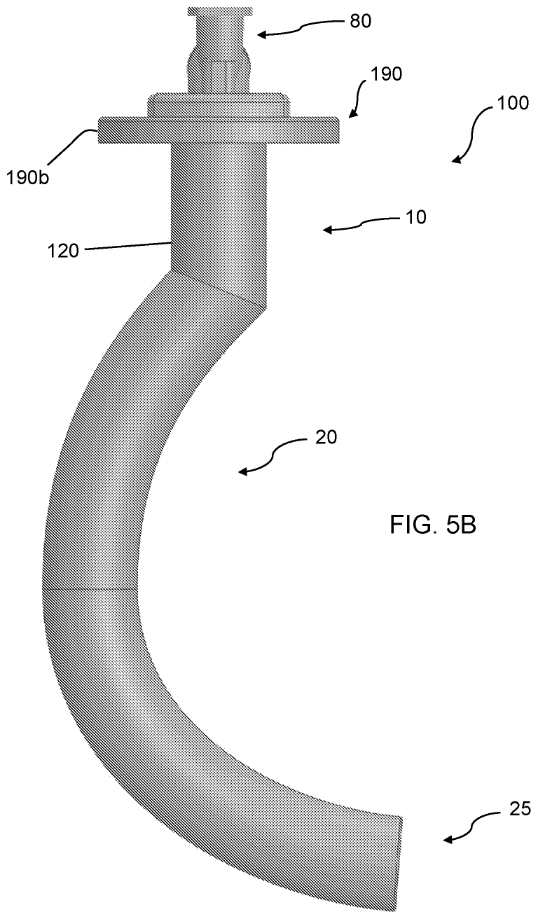

Embodiments of the present oral airway devices may be sized for use in pediatric patients (e.g., those requiring a 40-millimeter (mm) Guedel airway) up to extra-large adult patients (e.g., those requiring a 110-mm Guedel airway), including 50 mm, 60 mm, 70 mm, 80 mm, 90 mm, 100 mm, and 110 mm. These lengths are measured in the direction of the lengths SSL and CSL shown below in FIG. 5A.

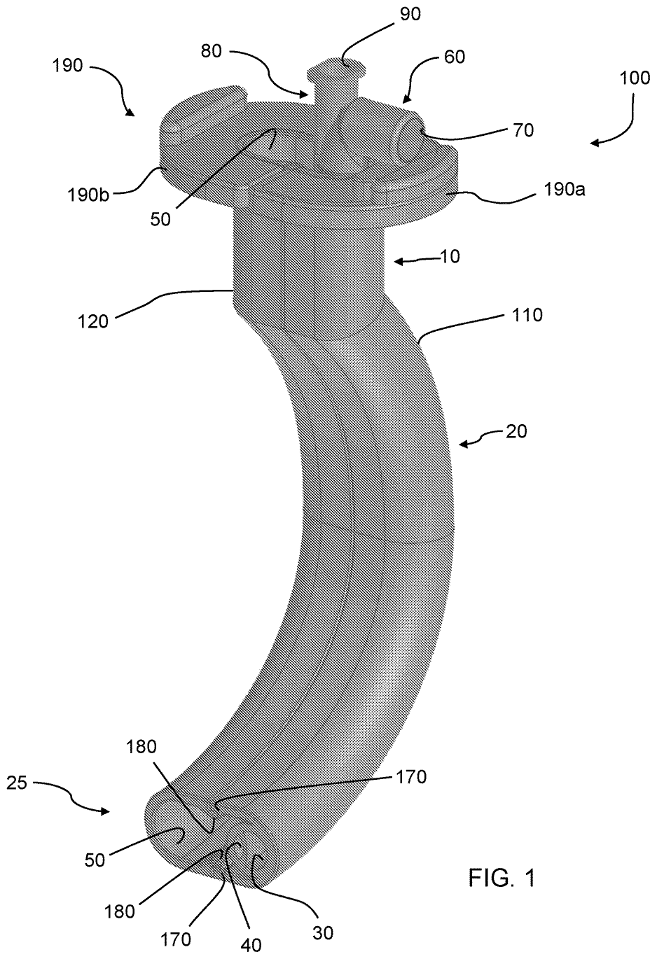

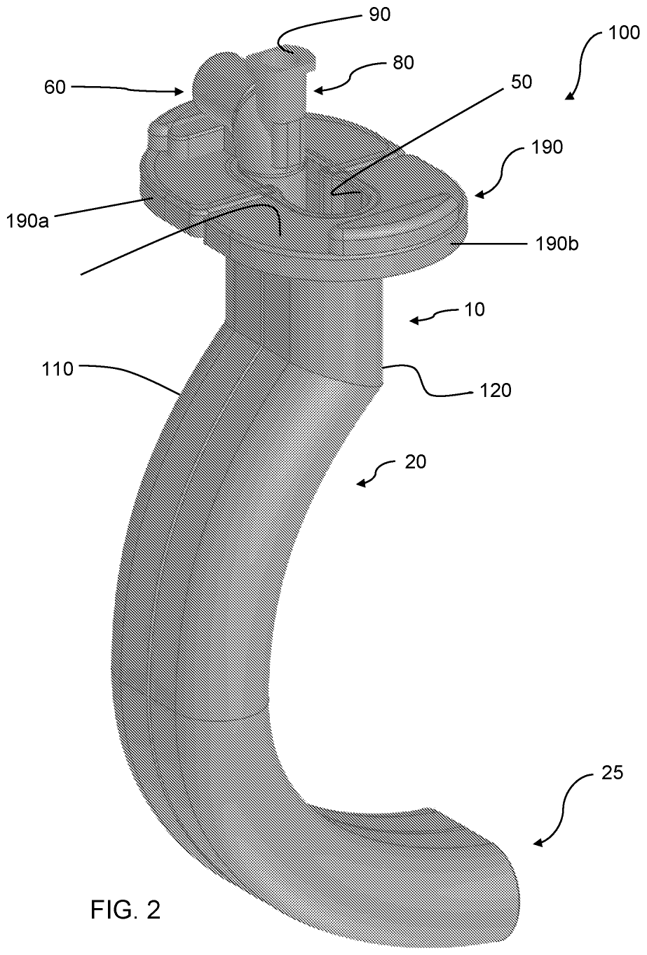

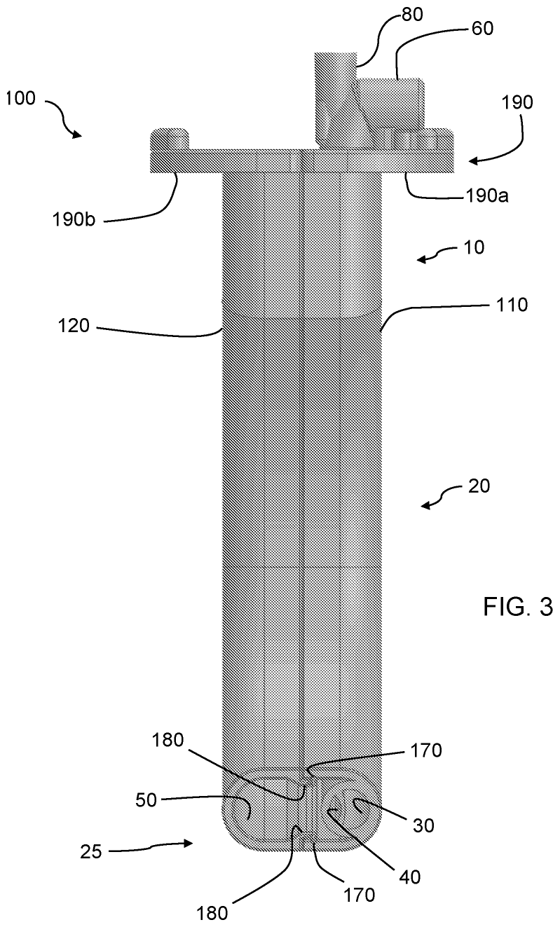

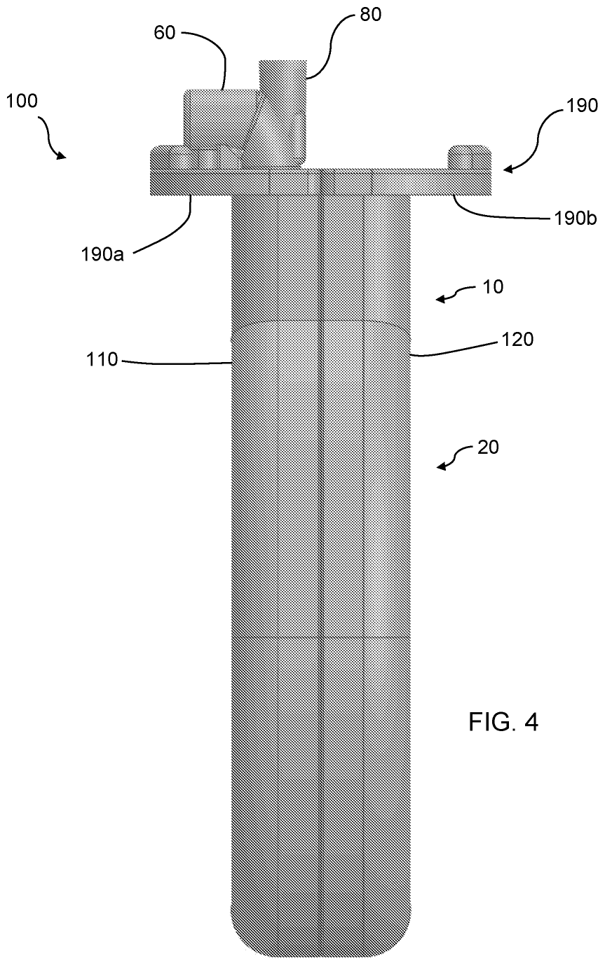

FIGS. 1 and 2 are perspective views of one embodiment of the present oral airway devices. FIGS. 3 and 4 are front and back views, respectively, thereof. The depicted device has a body 100 that includes a straight section 10, a non-straight (e.g., curved) section 20, a first lumen 30, a second lumen 40, a third lumen 50, a first connector 60, a second connector 80, and a flange 190 oriented at a non-zero (e.g., ninety-degree) angle to the straight section 10. In some embodiments, the curved section 20 has a pharyngeal curve. As reflected in these figures, the first connector 60 is oriented perpendicular to the straight section 10 of body 100, and the second connector 80 is oriented parallel to the straight section 10 of body 100.

Body 100 comprises a first body component 110 and a second body component 120 configured to be removably coupled to the first body component 110. The first lumen 30, the second lumen 40, and third lumen 50, which are visible at the distal end 25 of the body 100, are within the straight and curved sections of the body 100. More specifically, first and second lumens 30 and 40 are defined by first body component 110, and third lumen 50 is defined by the first and second body components in their coupled state, though a majority by volume of the third lumen 50 is defined by the second body component 120. The first connector 60, which is part of the first body component 110, has a first opening 70 in fluid communication with the first lumen 30. The second connector 80, which is also part of the first body component 110, has a second opening 90 in fluid communication with the second lumen 40. The first body component 110 includes grooves 170 into which portions of connector tabs 180 of the second body component 120 are positioned when the first and second body components are coupled. Each of the two grooves 170 of the first body component 110 extend substantially the length of the first body component 110 (excluding the first and second connectors). Similarly, each of the two connector tabs 180 of the second body component 120 extend substantially the length of the second body component 120. Each connector tab 180 of the second body component 120 is configured to (a) flex (inwardly) as the first body component 110 is coupled to the second body component 120 and (b) flex (outwardly) as the first body component 110 is uncoupled from the second body component 120. The flexibility of each connector tab 180 will be substantially elastic, which will allow the portion of each connector tab shaped in a complementary fashion to the groove 170 into which it will extend to extend into that groove when the two body components are placed into their coupled state.

The first body component 110 includes a first flange portion 190a oriented at a non-zero (e.g., ninety-degree) angle to the part of the straight section comprised by the first body component, and the second body component 120 includes a second flange portion 190b oriented at a non-zero (e.g., ninety-degree) angle to the part of the straight section comprised by the second body component. Flange 190 comprises first and second flange portions 190a and 190b. The first and second body components 110 and 120 are removably couplable to each other along majorities of their respective lengths; more specifically, the first and second body components are removably couplable to each other along the portions of their lengths extending from substantially the top (proximal-facing) surface of their respective flange portions to the substantially the distal ends of the components.

FIGS. 5A and 5B are right and left side views, respectively, of the oral airway device depicted in the preceding figures. In FIG. 5A, the first opening 70 of the first connector 60, which can be coupled to an oxygen source, faces the viewer and shows a portion of the first connector 60's lumen, which is in fluid communication with the first lumen 30. FIG. 5A also shows that the length SSL of the shortest portion of straight section 10 of the body 100 is less than the length CSL of the longest portion of curved section 20 of the body 100. This figure also shows that the length of the longest portion of straight section 10 is less than the length of the shortest portion of curved section 20. In FIG. 5B, the second connector 80, which can possess any configuration (such as a luer fitting, such as a luer lock fitting or a luer slip (or slip tip) fitting) suitable for coupling the connector to a carbon dioxide sample tube (or line), is visible near the top of the figure.

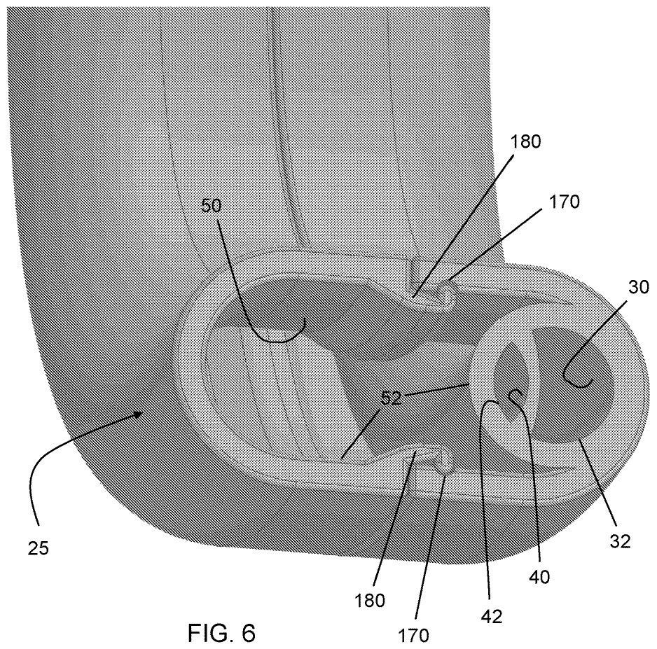

FIG. 6 shows an enlarged detail of a perspective view of the distal end 25 of the body 100 of the depicted oral airway device, showing aspects of the first lumen 30, the second lumen 40, and the third lumen 50. In at least some embodiments of the present oral airway devices, and as a person of ordinary skill in the art will appreciate from FIG. 6, in a cross-section of the body 100 that intersects and is substantially perpendicular to these lumens (which includes a cross-section of the body 100 taken along the straight section 10, and a cross-section of the body 100 taken along the curved section 20), the first lumen 30, second lumen 40, and third lumen 50 are bounded by respective first, second, and third perimeters 32, 42, and 52, with the first perimeter 32 being larger than the second perimeter 42, and the third perimeter 52 being larger than the first perimeter 32. In at least some embodiments of the present oral airway devices, and as a person of ordinary skill in the art will further appreciate from FIG. 6, the first, second, and third perimeters are non-circular and/or non-oval.

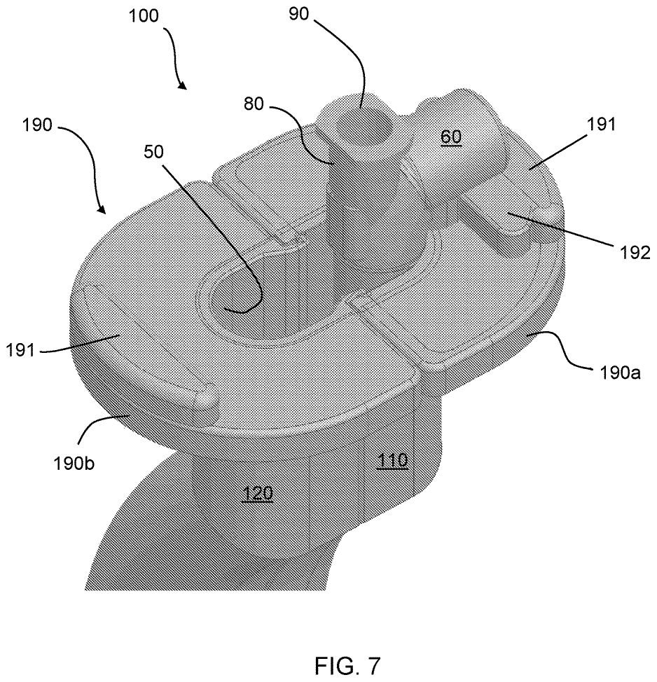

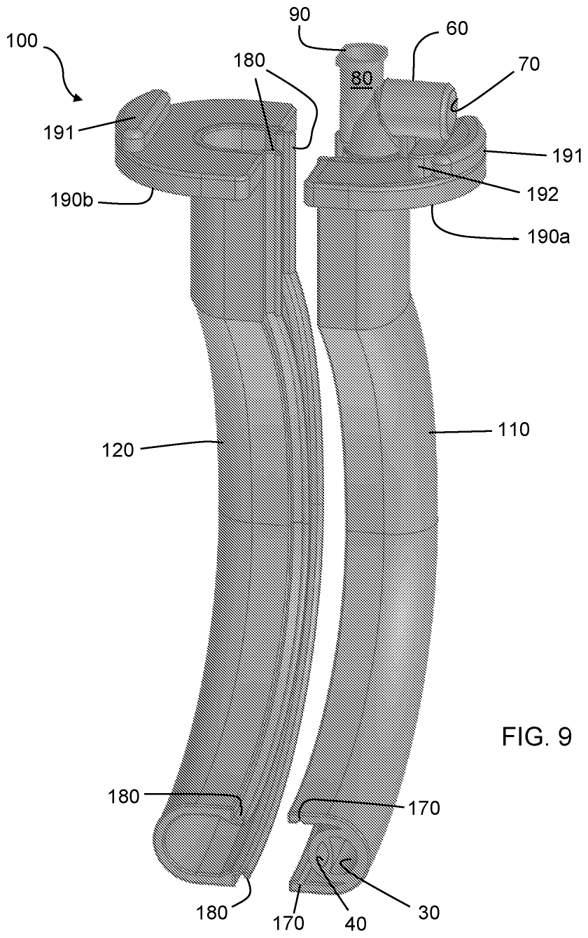

FIG. 7 shows an enlarged detail of a perspective view of a proximal portion of body 100 of the depicted oral airway, showing first flange portion 190a and second flange portion 190b of flange 190. In at least some embodiments of the present oral airway devices, and as a person of ordinary skill in the art will further appreciate from FIG. 7 as well as, e.g., FIGS. 1 and 9, the body 100 also includes a raised tab 191 on a proximal-facing side of each flange portion (190a and 190b). In other embodiments, the first flange portion 190a of flange 190 does not include such a raised tab. As shown in FIG. 7, as well as, e.g., FIG. 9, body 100 may also include a first connector-supporting portion 192, which is material that extends upward from the proximal-facing surface of the first flange portion 190a to underneath first connector 60; such a supporting portion should prevent any unwanted downward flexing of the first connector 60 when a practitioner uncouples the first body components 110 from the second body components 120 by applying forces to those body components, such as through opposing forces applied to the second connector 80 (and, thus, indirectly to the first connector 60) and to the raised tab 191 on the second flange portion 190b. In particular, a practitioner may uncouple the first and second body connectors from each other by placing (a) the forefinger and thumb of one hand under the second flange portion 190b and on the raised tab 191 (or the proximal-facing surface of the second flange portion 190b if no raised tab is present), respectively, of the second body component 120 and (b) the forefinger and thumb of the other hand under the first flange portion 190a and against the second connector 80, respectively, of the first body component 110, and pulling those gripped portions of the two body components away from each other.

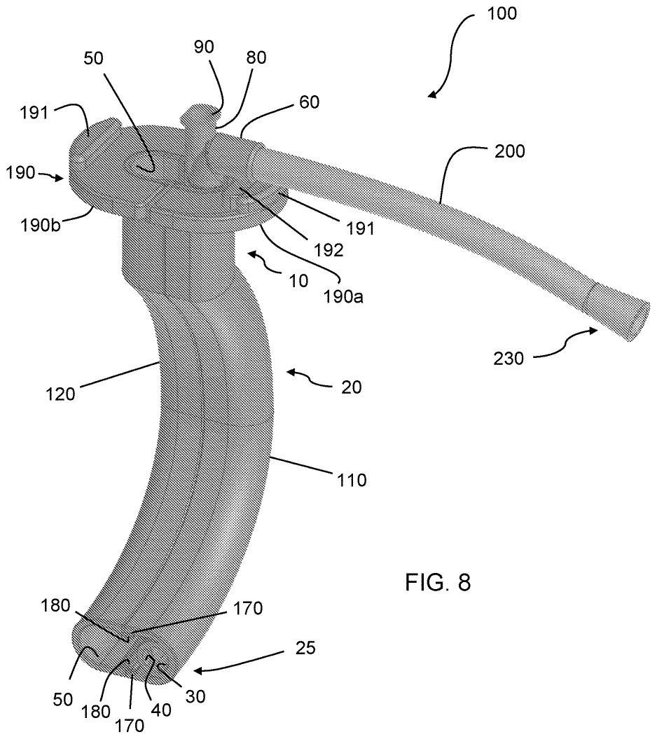

FIG. 8 shows the depicted oral airway device coupled to a length of tubing 200, one end of which is attached to the first connector 60. The length of tubing 200 includes a female connector 230 at its free end that may be connected to the male connector of a distinct length of tubing (not shown) that is connectable to an oxygen source (also not shown) for delivering oxygen to a patient. The first length of oxygen tubing 200 may have any suitable length, such as 15 to 20 cm in at least some embodiments (including any distance from 15 to 20 cm, including by increments of 1 cm, and shorter or longer distances in other embodiments), and may be connected to the first connector 60 of the body 100 in any suitable way known to a person of ordinary skill in the art, such as through a medical-grade adhesive. In the state shown in FIG. 8, the depicted oral airway device may be coupled to an oxygen source and inserted into a patient's mouth; such insertion can be accomplished in any conventional manner that a practitioner would otherwise use to insert into a patient's mouth a Guedel-style airway device. In embodiments of the present kits, the device may be in this state within the kit container (e.g., box or pouch). At least a portion of the body 100 distal to the flange portion 190 (e.g., along the straight section 10 thereof) may be configured to structurally resist damage due to a patient biting down thereon, and thus may each be characterized as having a bite guard.

In at least some embodiments of the present oral airway devices, each of the body components of the body 100 may be unitarily constructed, such as through injection molding, and may be made from any suitable material known to those of ordinary skill in the art, including medical grade plastics and plastic-like materials, such as polypropylene, polyethylene, polycarbonate, polyvinyl chloride, silicone, rubber, urethane, and the like. The material chosen should allow the constructed body components to be coupled and uncoupled without any portion of the connector tabs 180 of the second body component 120 breaking off during such coupling or uncoupling. As such, the material chosen should provide the connector tabs 180 with sufficiently flexibility and elasticity to accomplish such coupling and uncoupling, while also providing the balance of the body components with sufficient rigidity to enable their coupling and uncoupling.

FIG. 9 is a perspective view of the previously-depicted oral airway device, showing the body 100 thereof with its first body component 110 and second body component 120 in an uncoupled state. The connector tabs 180 of the second body component 120 are more visible in this figure than in previous figures.

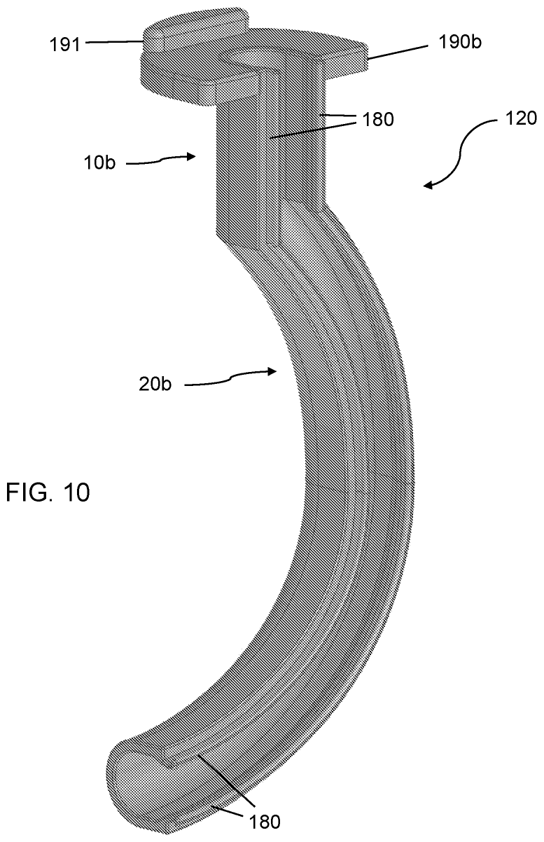

FIG. 10 shows the second body component 120 of the oral airway device depicted in FIG. 1. This figure shows the connector tabs 180 in even greater detail than FIG. 9. This figure (including in combination with other figures, such as, e.g., FIG. 3) also shows that, in at least some embodiments of the present oral airway devices, a majority by volume of the third lumen—formed by the coupled first and second body components—is defined by the second body component 110. As reflected in FIG. 10, the second body component 120 comprises a second part 10b of the straight section 10 and a second part 20b of the curved section 20.

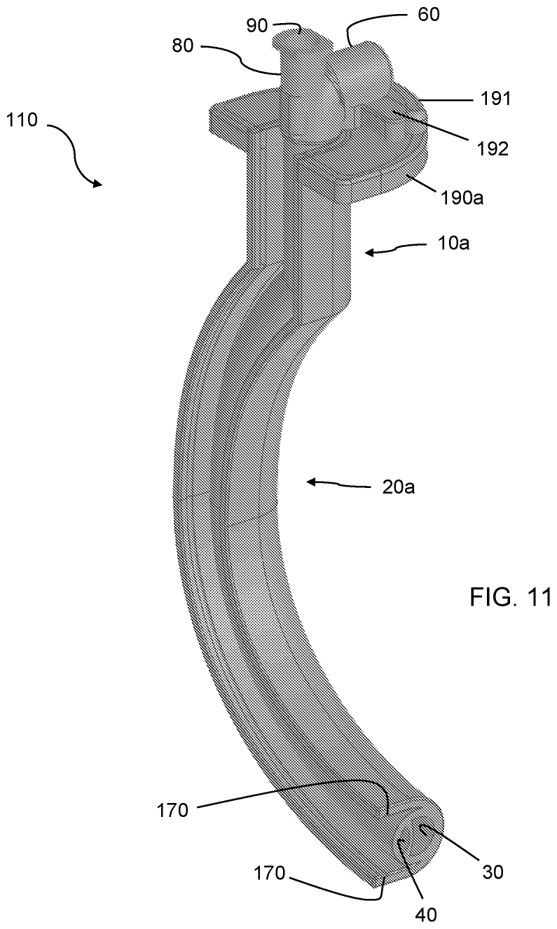

FIG. 11 shows the first body component 110 of the oral airway depicted in FIG. 1. As reflected in this figure, the first body component 110 comprises a first part 10a of the straight section 10 and a first part 20a of the curved section 20, and the first and second lumens 30, 40 are within the first parts 10a, 20a of the straight and curved sections 10, 20. FIG. 11 also shows one of grooves 170 in greater detail than other figures.

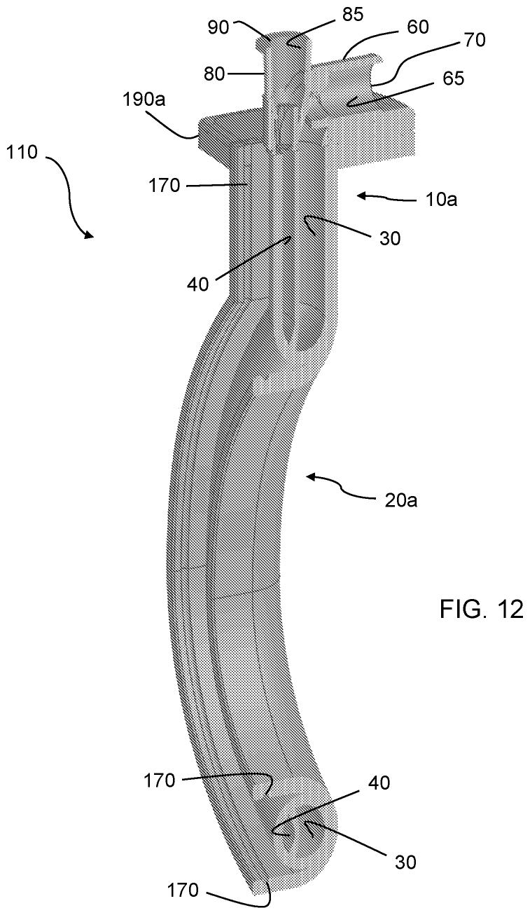

FIG. 12 depicts a cross-section of the first body component 110 taken along the length of the body and through portions of the first and second lumens 30, 40, showing the first and second lumens 30, 40 within the straight section 10 and the curved section 20 of the body. FIG. 12 also shows that lumen 65 of the first connector 60 is in fluid communication with the first lumen 30 and that the lumen 85 of the second connector 80 is in fluid communication with the second lumen 40.

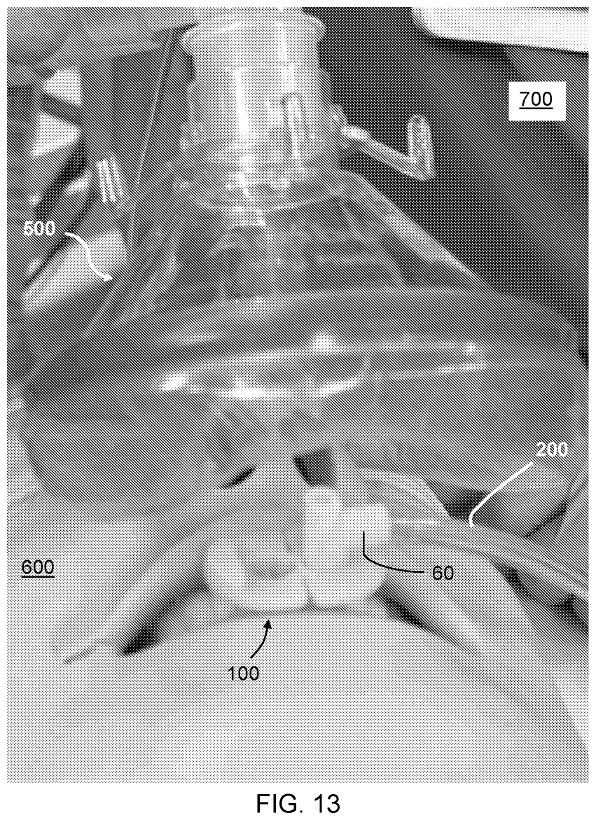

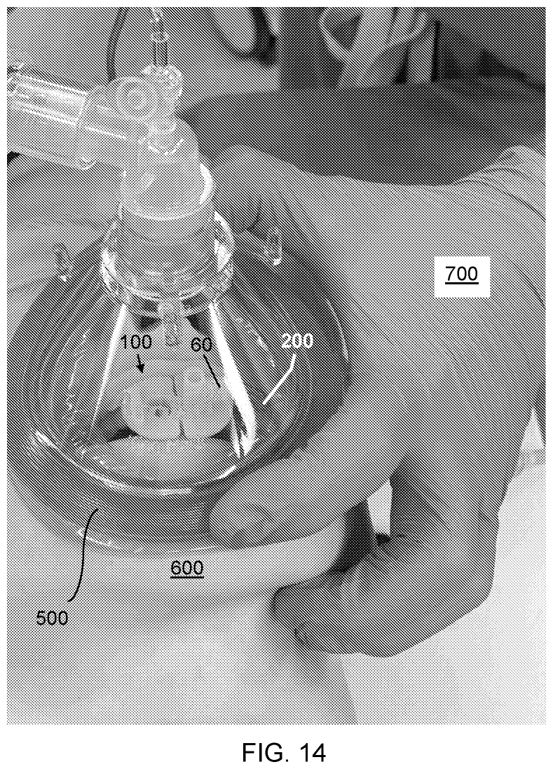

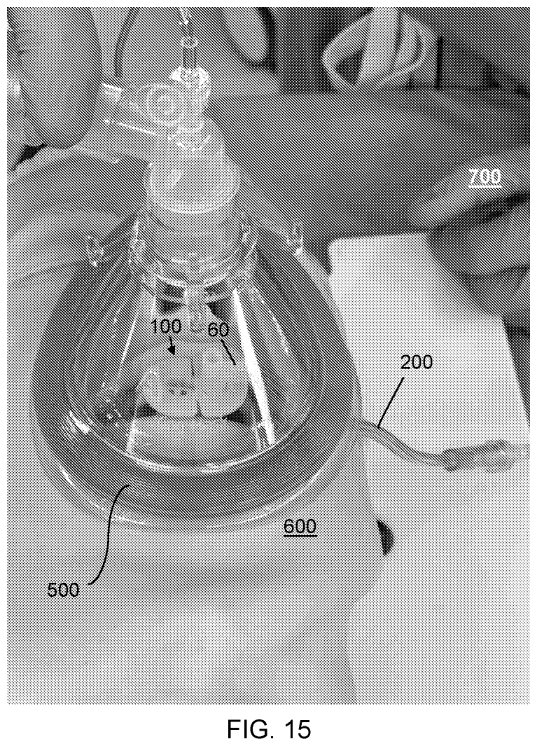

FIGS. 13, 14, and 15 show a mask 500 usable with a bag-valve-mask ventilation system (not shown) being placed into position over the nose and mouth of the head 600 of a mannequin, with body 100 of the depicted oral airway device coupled to the length of tubing 200 attached to the first connector 60 of the body 100 prior to sealing the mask over the mannequin's nose and mouth (FIG. 13) and after sealing by a practitioner 700 (FIG. 14) and with the mask in a sealed position but the practitioner's hand removed (FIG. 15). These figures reflect that a mask that is part of a conventional bag-valve-mask system will adequately seal over the nose and mouth of a patient with one of the present oral airway devices inserted in a patient's mouth and connected to a source of oxygen, thus allowing (as in some embodiments of the present methods) a practitioner to ventilate a patient while the patient is receiving oxygen through one of the present oral airway devices.

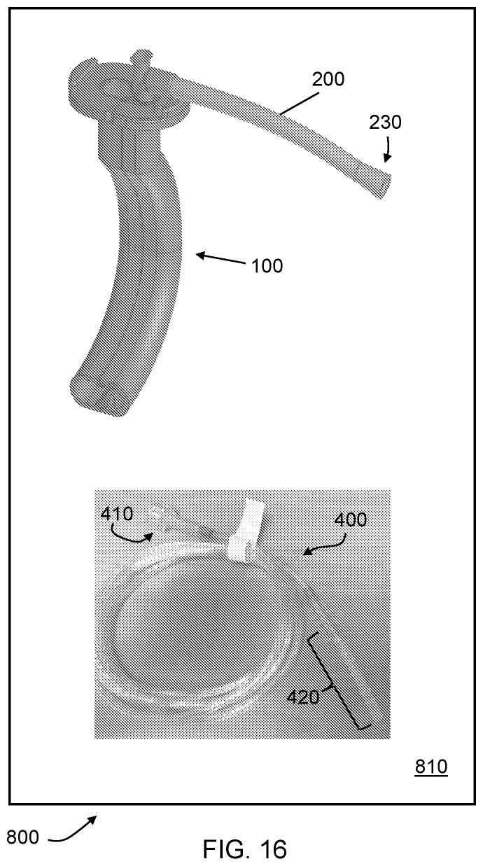

FIG. 16 schematically shows one of the present kits 800, which includes a container 810 in which is sealed at least one of the present oral airway devices (as reflected by the depiction of body 100, to which first length of oxygen tubing 200 is connected), and a second length of oxygen tubing 400 that may be used to connect the included oral airway device with an oxygen source. For example, second length of oxygen tubing 400 includes a female connector 410 that may be used to connect the tubing 200 to a source of oxygen at a wall or an e-cylinder (not shown), and, at another of its ends, the tubing 400 includes a male connector 420. The first length of oxygen tubing 200 includes a female connector 230 at its free end that may be connected to male connector 420 of tubing 400.

The above specification and examples provide a complete description of the structure and use of illustrative embodiments of this invention. Although certain embodiments have been described above with a certain degree of particularity, or with reference to one or more individual embodiments, those of ordinary skill in the art could make numerous alterations to the disclosed embodiments without departing from the scope of this invention. As such, the various illustrative embodiments of the present oral airway devices, kits, and methods are not intended to be limited to the particular forms disclosed. Rather, they include all modifications and alternatives falling within the scope of the claims, and embodiments other than those shown may include some or all of the features of the depicted embodiment. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus-or step-plus-function limitations, unless such a limitation is explicitly recited in a given claim using the phrase(s) “means for” or “step for,” respectively.

1. An oral airway device comprising:
a body that includes:
a straight section;
a curved section;
a first lumen within the straight and curved sections of the body;
a second lumen within the straight and curved sections of the body;
a third lumen within the straight and curved sections of the body;
a first connector having a first opening in fluid communication with the first lumen; and
a second connector having a second opening in fluid communication with the second lumen.

2. The device of claim 1, wherein the body comprises:
a first body component; and
a second body component configured to be removably coupled to the first body component.

3. The device of claim 1, wherein the body comprises:
a first body component comprising a first part of the straight section and a first part of the curved section; and
a second body component configured to be removably coupled to the first body component, the second body component comprising a second part of the straight section and a second part of the curved section.

4. The device of claim 1, wherein the body comprises:
a first body component comprising a first part of the straight section and a first part of the curved section, wherein the first and second lumens are within the first parts of the straight and curved sections; and
a second body component configured to be removably coupled to the first body component, the second body component comprising a second part of the straight section and a second part of the curved section, wherein a majority by volume of the third lumen when the first and second body components are coupled to each other is defined by the second body component.

5. The device of claim 4, wherein the first body component includes grooves into which portions of connector tabs of the second body components are positioned when the first and second body components are coupled.

6. The device of claim 4, wherein the first body component includes grooves in the first parts of the straight and curved sections, the second body component includes connector tabs on the second parts of the straight and curved sections, and, when the first and second body components are coupled, a portion of a connector tab is positioned in each groove.

7. The device of claim 5, wherein each connector tab is configured to flex as the first body component is coupled to the second body component.

8. The device of claim 7, wherein each connector tab is configured to flex as the first body component is uncoupled from the second body component.

9. The device of claim 8, wherein the body also includes a flange oriented at a non-zero angle to the straight section.

10. The device of claim 2, wherein a proximal portion of the first body component includes a first flange portion oriented at a non-zero angle to a first part of the straight section, and a proximal portion of the second body component include a second flange portion oriented at a non-zero angle to a second part of the straight section.

11. The device of claim 8, wherein a proximal portion of the first body component includes a first flange portion oriented at a non-zero angle to the first part of the straight section, and a proximal portion of the second body component include a second flange portion oriented at a non-zero angle to the second part of the straight section.

12. The device of claim 3, wherein the flange includes a second flange portion, and the body also includes a raised tab on a proximal-facing side of the second flange portion.

13. The device of claim 12, wherein the body also includes a raised tab on a proximal-facing side of the second flange portion.

14. The device of claim 13, wherein the first connector is oriented perpendicular to the straight section.

15. The device of claim 14, wherein the second connector is oriented parallel to the straight section.

16. The device of claim 15, wherein in a cross-section of the body taken along the straight section, the first and second and third lumens are bounded by respective first and second and third perimeters, the first perimeter is larger than the second perimeter, and the third perimeter is larger than the first perimeter.

17. The device of claim 16, wherein in a cross-section of the body taken along the curved section in a direction perpendicular thereto, the first and second and third lumens are bounded by respective first and second and third curved-section perimeters, the first curved-section perimeter is larger than the second curved-section perimeter, and the third curved-section perimeter is larger than the first curved-section perimeter.

18. The device of claim 17, further comprising a length of tubing having a first end that is attached to the first connector and a second end that is attached to a female connector.

19. An oral airway device comprising:
a body that includes:
a straight section having a straight section length;
a curved section extending from the straight section, the curved section having a curved section length that is greater than the straight section length;
a first lumen within the straight and curved sections of the body;
a second lumen within the straight and curved sections of the body;
a third lumen within the straight and curved sections of the body;
a first connector having a first opening in fluid communication with the first lumen;
a second connector having a second opening in fluid communication with the second lumen; and
a flange oriented at a non-zero angle to the straight section.

20.-33. (canceled)

34. An oral airway kit comprising:
an oral airway device having a body that includes:
a first lumen;
a second lumen;
a third lumen;
a first connector having a first opening in fluid communication with the first lumen; and
a second connector having a second opening in fluid communication with the second lumen; and
a length of tubing having a first end that is attached to the first connector and a second end that is attached to a female connector.

35.-66. (canceled)
